(19) United States
(12) Patent Application Publication
(10) Pub. No.: US 2002/0192303 A1
(43) Pub. Date: Dec. 19, 2002

(54) Treatment of side effects associated with alcohol consumption

(76) Inventors: Stefan Arver, Stockholm (SE); Olof Haglund, Stockholm (SE)

Correspondence Address: THORPE NORTH WESTERN, 8180 SOUTH 700 EAST, SUITE 200, P.O. BOX 1219, SANDY, UT, 84070, US

(21) Appl. No.: 10/067,733

(22) Filed: Jun. 1, 2001

(51) Int. Cl. A61K 33/00

(52) U.S. Cl. 424/722

(57) ABSTRACT

The present invention provides a composition and method for minimizing side effects associated with alcohol consumption. The composition includes an effective amount of a calcium antagonist, an osmo regulator which increases the alcohol clearance rate, and a fatty acid binder. Particularity a preferred embodiment comprises an effective amount of magnesium, and effective amount of taurine, and an effective amount of carnitine. The method of minimizing side effects associated with alcohol consumption includes administering an effective amount of the above enumerated composition to an individual in need of such an effect. The composition may be administered in various manners and times.

### PRIORITY DATA

This application claims priority to U.S. Provisional Patent Application serial No. 60/208,788, filed Jun. 1, 2000, which is incorporated herein by reference in its entirety.

### THE FIELD OF THE INVENTION

The present invention relates generally to a composition and method for alleviating or minimizing the undesirable side effects of alcohol over consumption. More particularly, it concerns a composition and method for preventing, minimizing, or alleviating the condition of a hangover.

### BACKGROUND OF THE INVENTION

For purposes of this disclosure, the terms alcohol, alcoholic beverages and ethanol may be used interchangeably unless otherwise indicated.

Consumption of alcohol in excessive amounts is well known to cause a variety of undesirable side effects which have become collectively known as a “hangover.” Symptoms of a “hangover” include, but are not limited to, headache, acid indigestion, queasiness or nausea, diarrhea, muscle aches, lethargy, and a general malaise.

It is generally believed that the undesirable side effects of alcohol are due to a variety of factors. One factor is accumulation of acetaldehyde in the body. Acetaldehyde is a product of the oxidation of ethanol by the enzyme alcohol dehydrogenase. Although acetaldehyde is metabolized at a greater rate than ethanol, acetaldehyde is more toxic, and has a more acute effect on the body. Acetaldehyde is further oxidized into acetic acid by the action of alcohol dehydrogenase, and aldehyde dehydrogenase.

Another factor contributing to the side effects of alcohol is disturbance of blood-sugar levels by promotion of glycogenolysis (breakdown of glycogen and release of glucose). Such a process elevates blood-sugar levels and eventually depletes liver glycogen. Therefore, chronic exposure to ethanol can result in acute hypoglycemia, especially if the individual is in a poor nutritional state, unless the liver glycogen can be replaced.

Another factor is fatigue from over exertion, often to the point of neuromuscular exhaustion. Such exertion most often results when the judgment of an individual is impaired and physical activity continues beyond the point at which an unimpaired person would stop. One effect of this intense activity is that anaerobic metabolism may occur, and as a result, significant levels of lactic acid may accumulate within the muscles.

Dehydration is another factor contributing to the symptoms of a hangover. Dehydration occurs as a result of the suppressive effect of ethanol on the anti-diuretic hormone, vasopressin, which elevates urine production.

Disturbance of the desirable bacterial life-forms occupying portions intestinal tract is a factor contributing to nausea. Additionally, severe nausea resulting in vomiting further contributes to dehydration.

Numerous remedies ranging from clinical to folklore have been proposed for preventing or treating a hangover. Various concoctions of food, beverage, and medicine are included. For example, one home remedy suggests eating a good meal before consuming alcohol, and then drinking two glasses of water and eating a banana after drinking alcohol and prior to going to sleep.

Another suggested treatment for reducing hangover symptoms is the administration of a composition containing activated charcoal. The charcoal supposedly absorbs a small amount of ethanol, along with large amounts of various alcohol metabolites which are believed to cause hangover symptoms.

Additional hangover treatments claim to provide relief by administering a composition containing a combination of ingredients that treat the various hangover symptoms. Particularly, these type of compositions generally include a pain reliever, such as acetaminophen, an antacid, a stimulant, such as caffeine, and an energy source such as sugar. Other treatments include specially designed substances which seek to attack and reduce the alcohol and its metabolic byproducts. One example of such a specially designed substance is a thiamine derivative which is meant to complex with acetaldehyde in the body, and therefore reduce its concentration and speed its elimination.

While the aforementioned methods present various products and methods for allegedly providing hangover relief, no satisfactory remedy has been discovered. Therefore, new compositions and methods continue to be sought.

### SUMMARY OF THE INVENTION

The present invention provides a method and composition for minimizing side effects associated with alcohol consumption. In one aspect of the invention, a composition for minimizing side effects associated with alcohol consumption includes a combination of a calcium antagonist, an osmo-regulator, and a fatty acid binder. In another aspect of the invention, a composition for minimizing side effects associated with alcohol consumption includes an effective amount of magnesium, an effective amount of taurine, and an effective amount of carnitine.

In yet another aspect of the invention, a method for minimizing side effects associated with alcohol consumption includes administering a composition comprising a calcium antagonist, an osmo-regulator, and a fatty acid binder to an individual in need of such a minimizing effect. In a further aspect of the invention, a method for minimizing side effects associated with alcohol consumption includes administering a composition comprising effective amounts of magnesium, taurine, and carnitine to an individual in need of such a minimizing effect.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying figures and claims, or may be learned by the practice of the invention.

### BRIEF DESCRIPTION OF THE DRAWINGS

FIG. 1 is a graphical comparison of the incidence of the symptom of headache between individuals who were administered a composition of the present invention and those who were administered a placebo in connection with drinking a certain amount of alcohol.

FIG. 2 is a graphical comparison of the time of reaction to a designated stimulus between individuals who were administered a composition of the present invention and those who were administered a placebo in connection with drinking a certain amount of alcohol.

FIG. 3 is a graphical comparison of the cognitive functions between individuals who were administered a composition of the present invention and those who were administered a placebo in connection with drinking a certain amount of alcohol. Particularly, the memory of individuals was tested for their ability to recall the names of particular cities.

FIG. 4 is a graphical comparison of the cognitive functions between individuals who were administered a composition of the present invention and those who were administered a placebo in connection with drinking a certain amount of alcohol. Particularly, the memory of the individuals were tested for their ability to recall the names of certain individuals.

FIG. 5 is a graphical comparison of the incidence and intensity of the symptom of shakiness on the day after drinking, between individuals who were administered a composition of the present invention and those who were administered a placebo in connection with drinking a certain amount of alcohol.

FIG. 6 is a graphical comparison of the change in blood pressure between individuals who were administered a composition of the present invention and those who were administered a placebo in connection with drinking a certain amount of alcohol.

FIG. 7 is a graphical comparison of the blood pressure profiles between the day of drinking and the day after drinking a certain amount of alcohol, for individuals who were administered a composition of the present invention.

FIG. 8 is a graphical comparison of the skin resistance on the day after drinking, between individuals who were administered a composition of the present invention and those who were administered a placebo after drinking a certain amount of alcohol.

### DETAILED DESCRIPTION

Before the present composition and method for minimizing or alleviating side effects associated with alcohol consumption is disclosed and described, it is to be understood that this invention is not limited to the particular process steps and materials disclosed herein, but is extended to equivalents thereof as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a” and, “the” include plural referents unless the context clearly dictates otherwise. Thus, for example, reference to a composition containing “a calcium antagonist” includes one or more calcium antagonists, reference to “an electrolyte replacement” includes reference to one or more of such electrolyte replacements, and reference to “the anti-oxidant” includes reference to one or more of such anti-oxidants.

In describing and claiming the present invention, the following terminology will be used in accordance with the definitions set forth below.

As used herein, “undesirable side effects” refers to any malady, unpleasant effect, or reduction in capacity of one or more normal physiological functions, which is associated with or results from, the consumption of alcohol, occurring either during or after alcohol consumption. Such a condition may be particularly acute when an excess or over consumption of alcohol occurs. By way of example, traditional symptoms of a hang over such as headache, nausea, light and sound sensitivity, etc. are undesirable side effects.

As used herein, an “effective amount,” and “sufficient amount” may be used interchangeably and refer to an amount of an ingredient which, when included in a composition, is sufficient to achieve an intended compositional or physiological effect. For example, a “sufficient amount” of a solvent would be the minimum amount needed to dissolve a target substance to a selected degree. Further, a “therapeutically effective amount” refers to an amount of a biologically active ingredient which is sufficient to achieve a desired and expected effect. The determination of an effective amount is well within the ordinary skill in the art of pharmaceutical, neutraceutical, herbaceutical, cosmetic, and medical sciences. See, for example, Meiner and Tonascia, “Clinical Trials: Design, Conduct, and Analysis,” Monographs in Epidemiology and Biostatistics, Vol. 8 (1986), incorporated by reference in its entirety.

As used herein, “pill form” refers to a number of solid dosage forms which are suitable for oral administration, including without limitation, tablets, capsules, powders, granules, etc.

As used herein, “magnesium” means any form of magnesium including organic or inorganic salts, complexes and chelates. Examples of forms of magnesium include but are not limited to: magnesium hydroxide, magnesium chloride, magnesium amino acid chelates, etc. Generally, the amount of magnesium which is contained in various magnesium compounds ranges from about 5% to about 60%.

As used herein, “taurine” means any form of taurine including analogs, derivatives, and acid addition salts. Taurine is listed in the Merck Index, entry no. 9241, 12^{th} ed. (1996).

As used herein, “carnitine” means any form of carnitine including analogs, derivatives, and acid addition salts. Carnitine is listed in the Merck Index, entry no. 1898, 12^{th} ed. (1996).

As used herein, “vitamin” includes any water or fat soluble vitamin which is necessary or helpful to the functioning of the human body.

As used herein, “energy replacing compound” means any substance which is capable of quickly imparting energy to a human body. Examples without limitation include simple carbohydrates such as mono- and disaccharides (e.g. glucose, fructose and sucrose).

As used herein, “antioxidant” means any compound which is capable of preventing or ameliorating oxidation.

As used herein, “pH adjuster,” “pH adjusting compound,” or the like means any compound which is capable of imparting a desired pH to the composition of the present invention, which is not harmful to humans when consumed in a quantity which is sufficient to impart the desired pH.

As used herein, “clearance rate” means the rate at which a substance is metabolized and removed from the body. In one aspect, the substance may be alcohol. In another aspect, the substance may be acetaldehyde.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

As an illustration, a concentration range of “about 0.1% to about 25% w/w” should be interpreted to include not only the explicitly recited concentration of about 0.1% to about 25% w/w, but also include individual concentrations and the sub-ranges within the indicated range. Thus, included in this numerical range are individual concentrations such as 2% w/w, 5% w/w, and 6% w/w, and sub-ranges such as from 1% w/w to 3% w/w, from 2% w/w to 6% w/w, from 8% w/w to 18% w/w, from 5% w/w to 20% w/w, etc. The same principle applies to ranges reciting only one numerical value.

Similarly, a range recited as “less than about 5.8% w/w” should be interpreted to include all of the values and ranges as elaborated above for the range of “from about 0.1% w/w to about 25% w/w.” Furthermore, such an interpretation should apply regardless of the breadth of the range or the characteristics being described.

The present invention is drawn to the discovery of a composition containing certain ingredients which are capable of minimizing side, reducing, or preventing, effects associated with the consumption of alcohol. The composition contains effective amounts of a calcium antagonist, an osmo-regulator which increases the clearance rate of alcohol, and a fatty acid binder. In one aspect, the calcium antagonist is magnesium, the osmo-regulator is taurine, and the fatty acid binder is carnitine.

Calcium is required for the proper functioning of numerous intracellular and extracellular processes, including muscle contraction, nerve conduction, hormone release, and blood coagulation. In addition, calcium ion plays a unique role in intracellular signaling, and is involved in the regulation of many enzymes.

Both extracellular and intracellular concentrations of calcium are tightly regulated through bi-directional calcium transport across the plasma membrane of cells, and by the membranes of intracellular organelles such as the endoplasmic reticulum, the sarcoplasmic reticulum of muscle cells, and the mitochondria. The transport of calcium out of the cytoplasm of cells and into these various compartments, along with the high degree of calcium protein binding maintains the concentration of ionized calcium in the cytoplasm in the micromolar range.

Unfortunately, one of the effects of alcohol consumption is an intracellular accumulation of calcium, especially in nerve cells. Such an accumulation overloads a cell with calcium and simultaneously deprives the extracellular region from a proper calcium concentration. Thus proper cellular function is disrupted.

The calcium antagonist ingredient of the present invention, such as magnesium, acts to normalize intracellular and extracellular calcium concentrations. Particularly, magnesium lowers intracellular calcium concentrations and raises extracellular calcium concentrations by facilitating calcium movement out of the cell. The normalizing of calcium concentrations restores cell functioning, and in so doing reduces the side effects of alcohol consumption, such as shakiness, and impaired cognitive abilities.

Another physiological effect of alcohol consumption is elevated blood pressure. Elevated blood pressure, or hypertension, is a significant cause of damage to the circulatory system, and contributes to headaches. Particularly, as blood pressure increases, so does the incidence of arteriosclerosis. Additionally, hypertension contributes to headaches by elevating pressure in the capillaries of the head. Calcium antagonists, such as magnesium lower blood pressure. By counteracting the blood pressure elevating action of alcohol, the side effects resulting from elevated blood pressure are reduced or eliminated.

In addition to interrupting proper calcium concentrations, the presence of alcohol in the body disrupts normal cell osmolarity in general. Thus, cellular operation dysfunctions to a certain degree causing fluid, electrolyte, protein, and nutrient imbalances. Additionally, as recited above, the metabolism of alcohol into acetaldehyde triggers inflammatory responses through the body.

Taurine is a conditionally essential amino acid for adult humans which is found throughout the body, but is particularly prevalent in the heart, eye, muscle, and brain tissues. Taurine is known to function in regulating and normalizing cell osmolarity. As such, taurine is instrumental in directing cell metabolism and controlling fluid and electrolyte distribution.

Additionally, taurine is a key detoxification substance. Particularly, taurine facilitates the decomposition of varying compounds and increases the rate at which certain substances are metabolically cleared.

These properties make taurine an effective osmo-regulator, for reducing or eliminating side effects associated with alcohol consumption. Particularly, taurine counteracts the negative effects of alcohol in disrupting cell osmolarity, thus restoring and maintaining normal cellular function. Additionally, taurine speeds up the processing of alcohol through the body, and effectively increases its clearance rate. Thus alcohol and its degradation products, such as acetaldehyde, are more quickly eliminated. In turn, the side effects of alcohol are reduced, and an intoxicated person becomes sober more quickly.

Alcohol has a particular propensity for binding with fatty acids in the body. Therefore, when introduced in significant quantities, the alcohol will complex with available fatty acids to form toxic esters. Additionally, because of the propensity for fatty acids to bind to alcohol, they become unavailable to perform their normal metabolic duties. Areas particularly affected by a lowered instance of free fatty acids are the striated and cardiac muscles.

Carnitine is a fatty acid binder which competes with alcohol. Additionally, carnitine is an essential cofactor in fatty acid metabolism. By competing with alcohol for fatty acid binding, carnitine both reduces the amount of toxic esters which are formed, and has no detrimental effect on the transport and use of fatty acids in the striated and coronary muscle cells.

It has been found that magnesium, taurine and carnitine, when administered concomitantly in therapeutically effective amounts, and in appropriate ratios, are more beneficial than administering such ingredients separately.

The type and amounts of magnesium, taurine, and carnitine must be effective for their intended purpose, but may vary depending on the desired characteristics of the final formulation. Any forms of magnesium, taurine, and carnitine are acceptable. In one aspect, a mixture of magnesium hydroxide, L-taurine, and L-carnitine may be used. However, complexes or chelates of magnesium with L-taurine and/or L-carnitine may also be used. The only limitation as to form is that these ingredients must be bioavailable and functional for their intended use.

In the following description relative amounts of magnesium, taurine, and carnitine in a composition are described in terms of parts by weight in order to show the relative ranges of each ingredient and ratios of one ingredient to another. When determining dosage forms the term parts by weight may be converted into weight units such as milligrams or grams, as the case may be, and can be readily determined by one having ordinary skill in the art.

In one aspect, the amount of magnesium may be from about 15 to about 1000 parts by weight. In another aspect, the amount of magnesium may be from about 100 to about 500 parts by weight. In one aspect, the amount of taurine may be from about 50 to about 1500 parts by weight. In another aspect, the amount of taurine may be about 200 to 600 parts by weight. In one aspect, the amount of carnitine may be from about 1 to about 400 parts by weight. In another aspect, the amount of carnitine may be from about 100 to about 300 parts by weight.

Additional active ingredients may be added to the composition in order to impart additional desired effects. Such ingredients include, but are not limited to, electrolyte replacing compounds, energy replacing compounds, vitamins, antioxidants, zinc, and zinc compounds, and pH adjustment compounds.

Inactive ingredients may be added as required to impart desired characteristics to the final dosage formulation. Examples of inactive ingredients include, but are not limited to, flavorants, binders, preservatives, and fillers.

Electrolyte replacements may be any compound, or combination of compounds, known for use in replacing electrolytes. In one aspect, electrolyte replacers include but are not limited to NaCl, and KCl, or a combination thereof. While the amount of electrolyte replacer may be any amount required to achieve a desired effect, in one aspect, the amount may be from about 25 to about 400 parts by weight. In another aspect, the amount may be from about 100 to about 300 parts by weight.

Energy replacing compounds may be any compound, or combination of compounds known for use in providing quick energy to the body, such as simple carbohydrates. In one aspect, energy replacers include but are not limited to sucrose, glucose and fructose, or combinations thereof. While the amount of energy replacement compound may be any amount required to achieve a desired effect, in one aspect, the amount may be from about 500 to about 4000 parts by weight. In another aspect, the amount may be from about 2000 to about 3000 parts by weight.

Suitable vitamins for inclusion in the composition of the present invention in order to prevent vitamin deficiency, include any vitamins required to provide a desired effect. In one aspect, vitamins include but are not limited to, any vitamin B complex (including niacin), folic acid, vitamin E, and vitamin C. While included for its antioxidant properties, vitamin C may also serve as an effervescence causing ingredient when combined with an appropriate base. While the amount of vitamin C may be any amount required to impart a desired effect, in one aspect, such an amount may be from about 25 to about 200 parts by weight. In another aspect, the amount may be from about 50 to about 150 parts by weight. Additionally, while the amount of any B vitamin complex may be any amount required to impart a desired effect, in one aspect, such an amount may be from about 5 to about 25 parts by weight. In another aspect, such an amount may be from about 10 to about 15 parts by weight. Finally, while the amount of folic acid may be any required to impart a desired effect, in one aspect, the amount may be from about 0.01 to about 1 parts by weight. In another aspect, the amount may be from about 0.1 to about 0.5 parts by weight.

The antioxidant may be any compound, or combination of compounds which are required to provide a desired antioxidant effect. In one aspect, antioxidants may include without limitation, all forms of vitamin C, such as sodium ascorbate, and vitamin E, such as gamma, or d-alpha tocopherol. While the amount of antioxidant may be any amount required to impart a desired effect, in one aspect, the amount may be from about 50 to 150 parts by weight.

Compounds which adjust pH may be any compound, or combination of compounds suitable for ingestion which is capable of providing a desired pH. However, in one aspect, the pH adjusting compound may be sodium bicarbonate. In addition to its pH adjusting properties, sodium bicarbonate may serve as an effervescence causing ingredient. While the pH adjusting compound may be present in any amount required to achieve a desired effect, in one aspect the amount may be from about 100 to about 300 parts by weight. In another aspect, the amount may be from about 150 to about 250 parts by weight.

The mineral zinc, or compounds thereof which make zinc bioavailable, such as ZnCl₂, ZnBr₂, or zinc chelated compounds, may be included in the present composition. As the enzyme alcohol dehydrogenase is a zinc metalloenzyme, zinc supplementation allows for a more rapid formation of alcohol dehydrogenase. Further, as zinc may be a limiting factor in the formation of alcohol dehydrogenase, zinc supplementation may allow for greater amounts of alcohol dehydrogenase to be produced by the body. While zinc may be present in any amount required to achieve a desired effect, in one aspect such an amount may be from about 1 to 50 parts by weight. In another aspect, the amount may be from about 20 to about 40 parts by weight.

The composition of the present invention may take various administration forms. In one aspect, the composition is administered in an oral dosage formulation. Oral dosage formulations include, but are not limited to tablets, capsules, powders, and liquids, and may be made by any method know by those ordinarily skilled in the art of making oral dosage formulations.

The solid dosage forms may be administered either directly, for example, by swallowing or chewing a tablet, or indirectly, by dispensing the solid form into an aqueous solution, and then consuming. If the solid form which is dispensed into a liquid contains effervescence causing ingredients, then the liquid may be effervescent.

Alternatively, a liquid dosage may be formed by skipping the steps required to make a solid dosage formulation, and initially adding the desired ingredients to an aqueous solution. Such liquids may be made effervescent by using any method well known in the art of beverage making.

The dosage regimen for administering the composition of the present invention includes administration before, during, or after alcohol consumption. In one aspect, the regimen includes consuming 3 doses of the composition. A first dose may be administered before alcohol consumption begins, a second does may be administered shortly after alcohol consumption ceases, and a third dose may be administered within about 8 to 12 hours thereafter. In one aspect, all three doses may be administered within a twenty hour period.

The example provided below is illustrative of only one embodiment of a composition of the present invention. While the combination of ingredients may be preferred, no limitation thereto is to be inferred.

### EXAMPLE

The ingredients in the following table, were combined in the amounts specified into an oral dosage formulation which was administered as indicated in the description of the clinical trial below. Generally speaking, the numerical value of ingredients measured in units of parts by weight are approximately equal to the numerical value of those ingredients when measured using standard weight units, such as mg, in a dosage composition.

| Ingredient type | Ingredient | Amount in mg |
|---|---|---|
| Electrolyte | NaCl | 100 |
| Replacement | | |
| Electrolyte | KCl | 100 |
| Replacement | | |
| pH Adjustment | Ca(HCO₃)₂ | 300 |
| Energy Replacement | Fructose | 500 |
| Vitamins | Vitamin B₁ | 1.2 |
| Vitamins | Vitamin B₂ | 1.6 |
| Vitamins | Niacin | 18 |
| Vitamins | Vitamin B₆ | 2 |
| Vitamins | Vitamin B₁₂ | .001 |
| Vitamins | Folic Acid | .200 |
| Calcium Antagonist | MgOH | 100 |
| Antioxidant | Vitamin E | 50 |
| | (d-alfa tocoferol) | |
| Antioxidant | Vitamin C | 100 |
| | (sodium ascorbate) | |
| Osmo Regulator | Taurine | 400 |
| Fatty Acid Binder | L-Carnitine | 20 |
| Zinc | ZnCl₂ | 10 |

A randomized double blind study was conducted on 28 subjects who were social drinkers between the ages of 20 and 50 having generally good health. Half of the subjects were given the above listed composition, and the other half were randomly administered a placebo. Administration of the composition and placebo occurred in three equal doses. The first administration occurred at the commencement of alcohol consumption, the second occurred after alcohol consumption was completed and prior to sleeping, and the third administration occurred after awaking the next morning.

Prior to the administration of alcohol, each subject was tested for various conditions including blood pressure, shakiness, headache, skin resistance, and reaction time. Additionally, during intoxication each subject was given memory testing. These conditions were then reevaluated the morning after intoxication.

The alcohol used in the study was Vodka having 40% alcohol by volume. Each male participant was administered 0.5 mL alcohol per kg of body weight. Each female participant was administered 0.4 mL of alcohol per kg of body weight. Blood alcohol contents were measured approximately 3 hours after the consumption of alcohol, and were uniformly between 0.8 and 0.9 mg/ml.

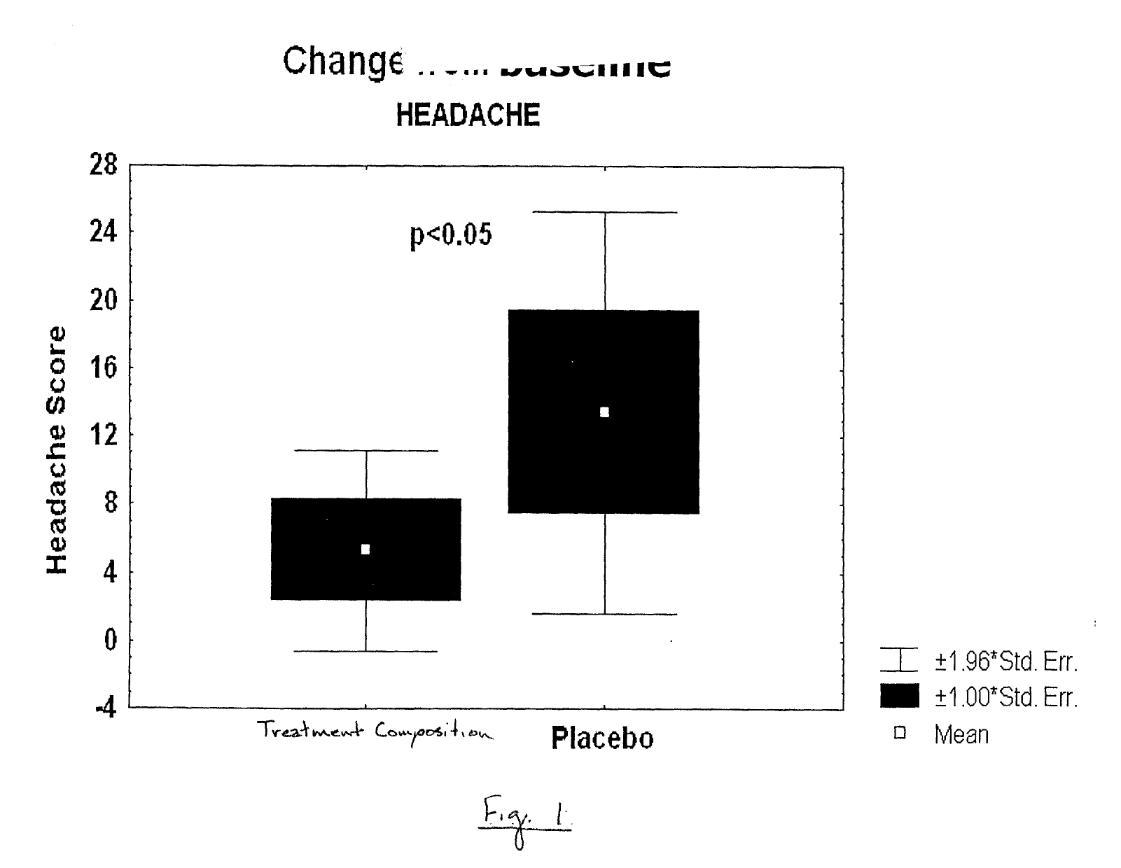

FIG. 1 shows the results of the headache test performed on the subjects the morning after drinking using a visual analog scale. As can be seen, the incidence and intensity of headache was significantly lower for those who received the composition of the present invention, than for those who received the placebo.

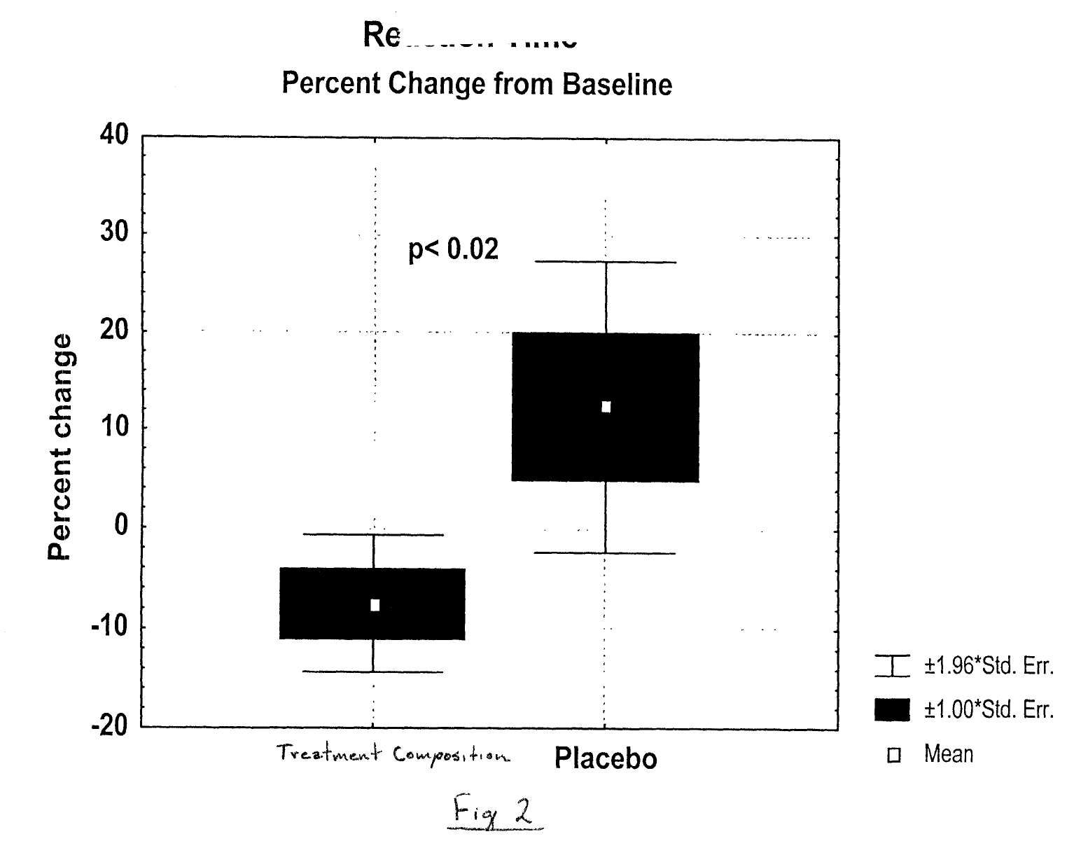

FIG. 2 shows the results of the reaction time tests which were conducted. This test was performed before the consumption of alcohol in order to determine a baseline value. The test was conducted using a standardized test involving a centimeter graded piece of paper which was dropped from a vertical position. The centimeter scale was calibrated to a time scale. The paper was held in front of a subject between the subjects open thumb and forefinger and dropped. The measurement of where the subject caught the dropped paper was measure to the closest 0.1 cm, and reaction time was then calculated. The reaction time change was repeated the morning after drinking. The results of FIG. 2 show that the subjects who received the treatment composition had reaction times significantly better than those who received the placebo.

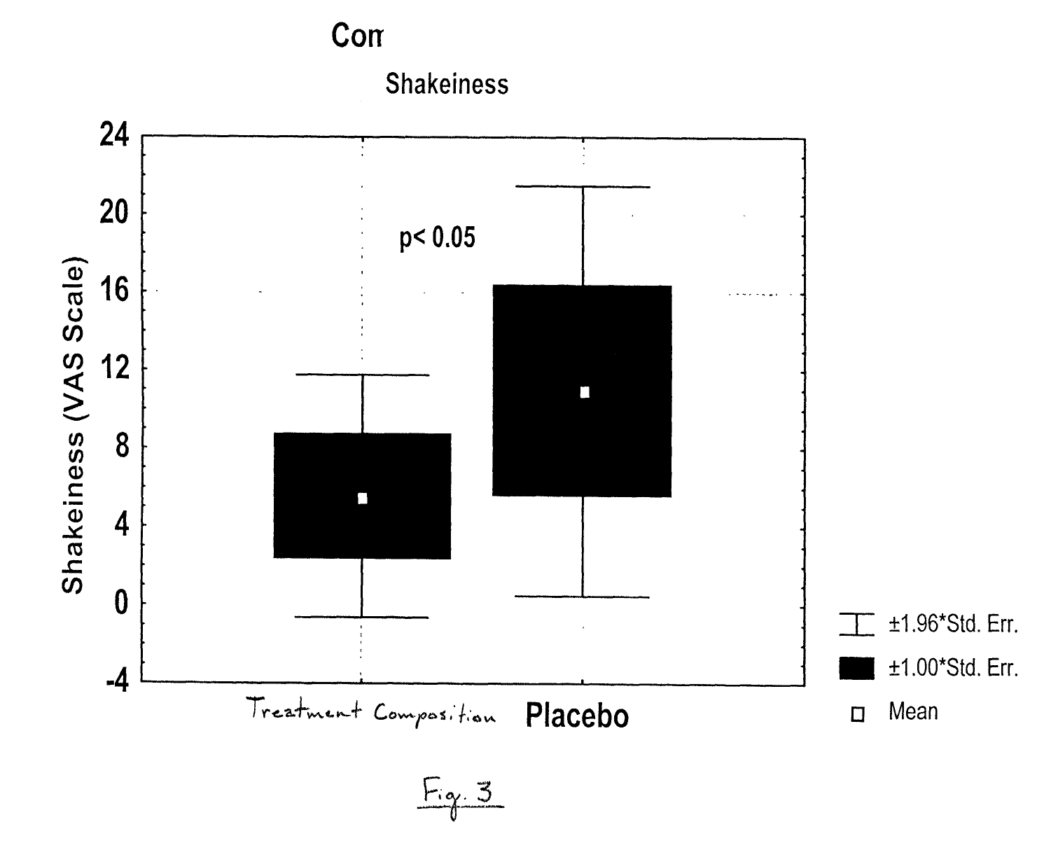

FIG. 3 shows the results of the shakiness test performed on the subjects the morning after drinking using a visual analog scale. As can be seen, the incidence and intensity of shaking was significantly lower for those who received the composition of the present invention, than for those who received the placebo.

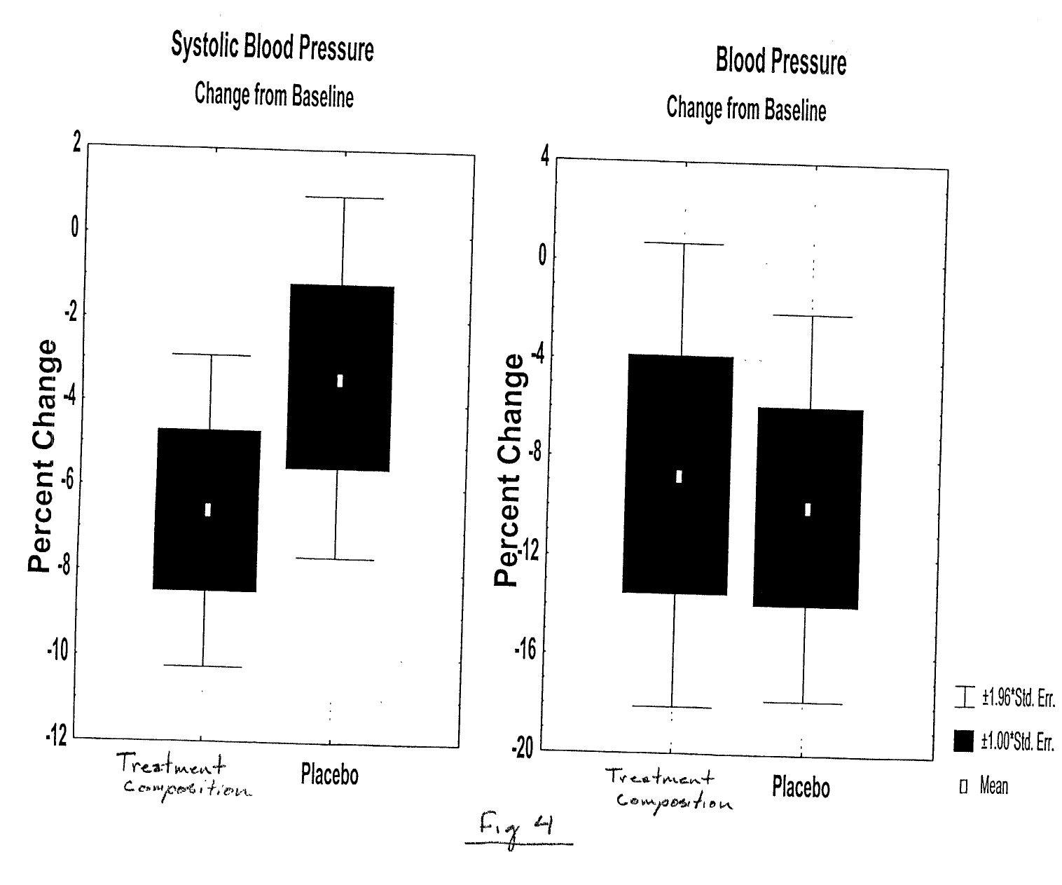

FIG. 4 shows the results of the blood pressure test performed. The blood pressures of the subjects was tested prior to alcohol consumption in order to establish a baseline value. Blood pressure was the re-evaluated the morning after drinking. FIG. 4 also shows that systolic blood pressure value was significantly lower for subjects who received the composition of the present invention, than for those who received the placebo. Additionally, FIG. 4 shows that the diastolic blood pressure value was about the same for subjects in both categories.

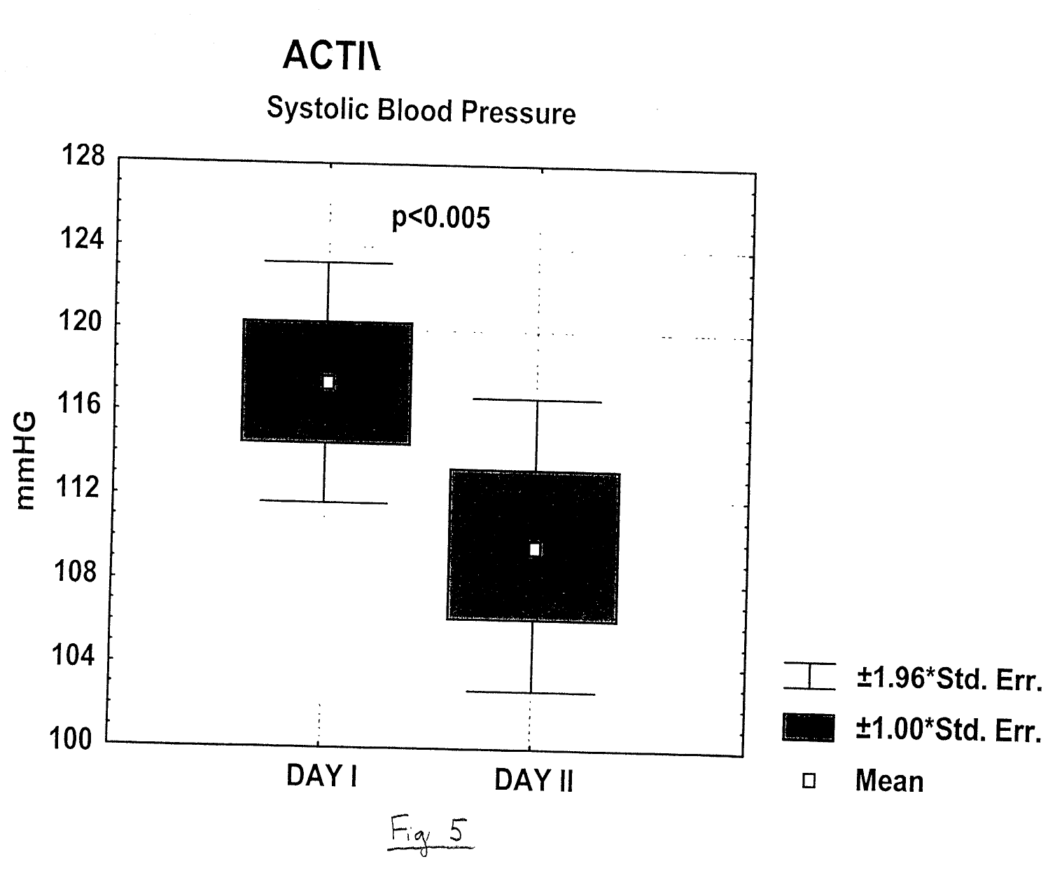

FIG. 5 further shows specific systolic blood pressure readings for individuals who received the composition of the present invention. Particularly, FIG. 5 shows that the systolic blood pressure readings for these individuals was lower on the morning after alcohol consumption using the composition of the present invention, than when taken before commencing alcohol consumption.

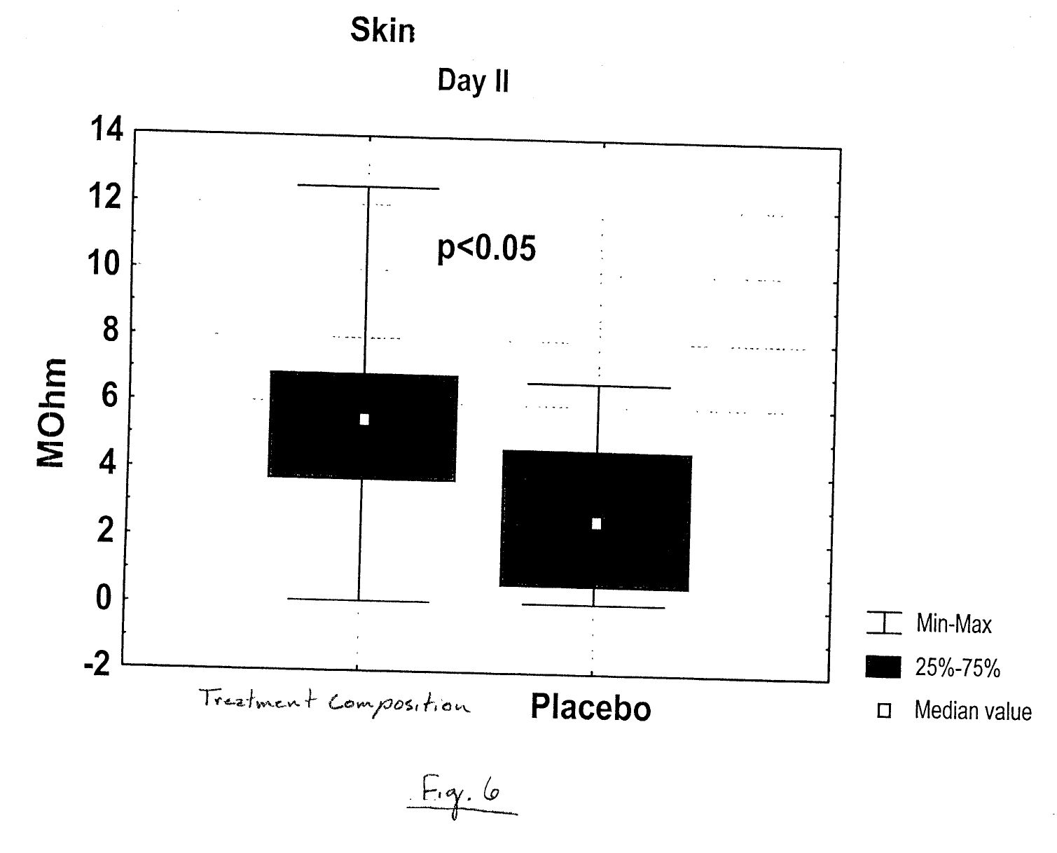

FIG. 6 shows the results of the skin resistance tests. These tests were conducted using an Ohm device attached to the inner forearm of each subject. The Ohm device contained Ag/AgCl electrodes which were applied to the skin approximately 5 cm above the styloid process, with a distance of about 10 cm between the electrodes. A voltage was then applied and the resistance measured.

Testing was conducted in this manner prior to the commencement of alcohol consumption in order to establish a baseline value. Skin resistance was then re-evaluated the morning after drinking in order to determine the incidence and extent of sweating or perspiration due to hangover. As can be seen the subjects who received the treatment composition of the present invention had a lower incidence of sweating than those receiving the placebo. This result is shown in FIG. 6, as skin resistance for the subjects who received the composition of the present invention is significantly higher than for those who received the placebo. The increased resistance indicates less moisture on the skin.

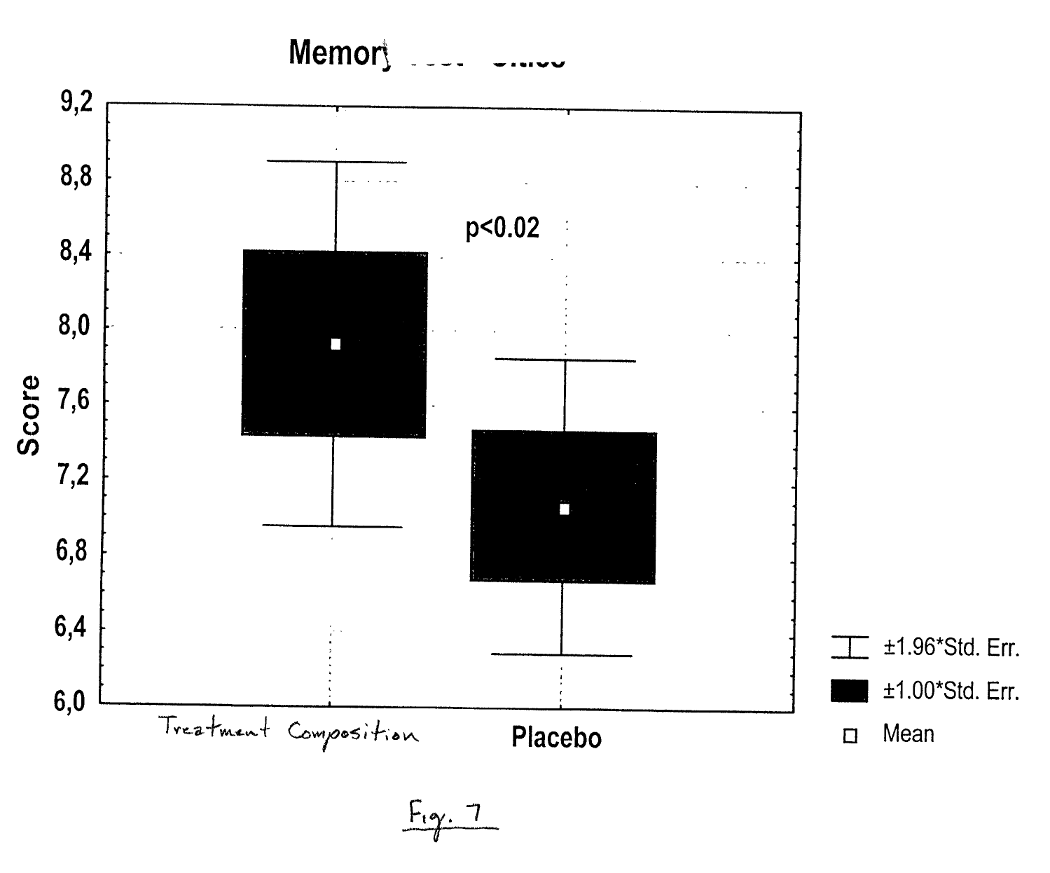

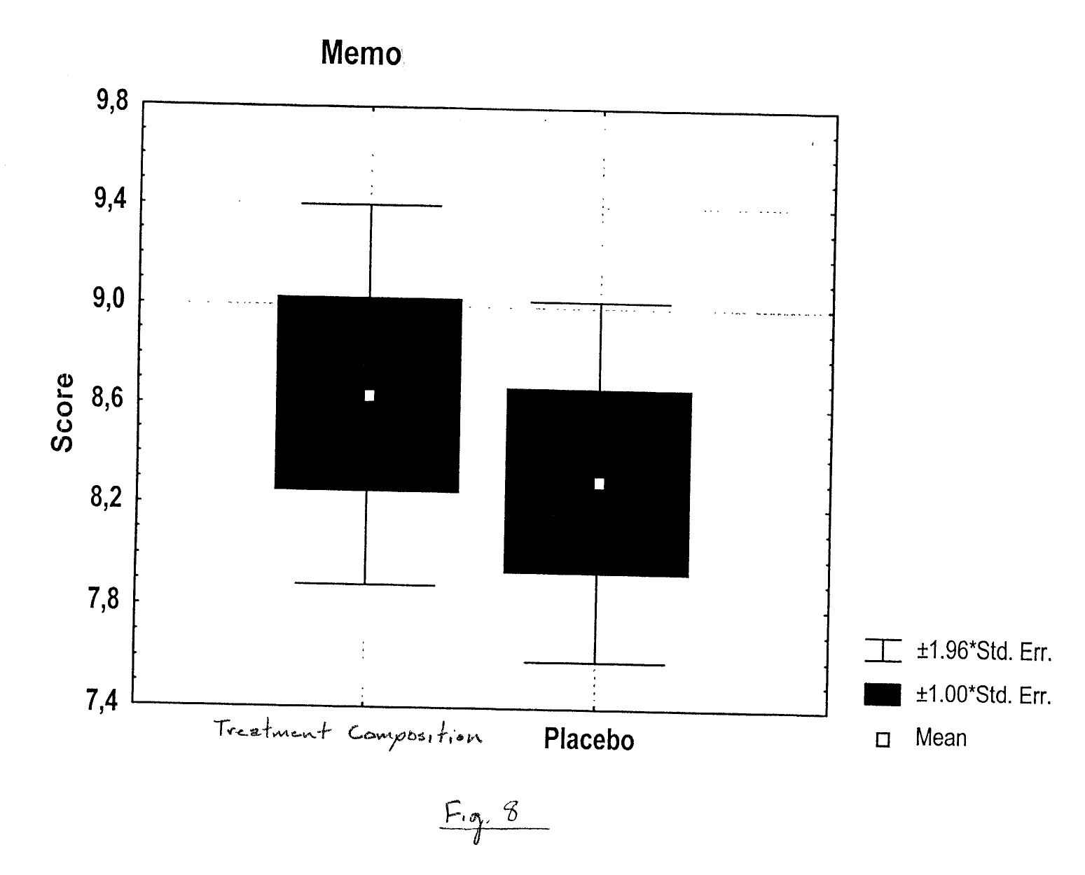

FIGS. 7 and 8 shows the results of the memory tests conducted on individuals. FIG. 7 shows the results for the names of cities, and FIG. 8 shows the results for the names of people. The memory test was conducted during intoxication and the morning after drinking. In each memory test, the subjects were shown a list with 11 numbers, 10 personal names, and 9 city names respectively for a duration of 1 minute. After a waiting period of 5 minutes, the subjects were allowed 1 minute to write down everything that they could remember. Both FIGS. 7 and 8 show that the subject who received the treatment composition had superior memory capabilities, and thus cognitive function, than those who received the placebo. This was true both during intoxication, and on the morning after.

As can be seen, the results of the study lead to the conclusion that the side effects of alcohol consumption may be minimized by administration of the composition of the present invention. Particularly, blood pressure, headache, cognitive abilities, incidence of sweating, shakiness, and reaction time are all improved in individuals receiving the composition as compared to those individuals receiving the placebo.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. An oral dosage treatment composition for ameliorating undesirable side effects due to alcohol consumption comprising:
a mixture of a therapeutically effective amount of a calcium antagonist, a therapeutically effective amount of an osmo-regulator, and a therapeutically effective amount of a fatty acid binder.

2. The composition of claim 1, wherein the calcium antagonist is a magnesium salt, complex, or chelate.

3. The composition of claim 1, wherein the osmo-regulator is taurine.

4. The composition of claim 1, wherein the fatty acid binder is carnitine.

5. The composition of claim 1, wherein the calcium antagonist is a magnesium salt, complex, or chelate, the osmo-regulator is taurine, and the fatty acid binder is carnitine.

6. The composition of claim 5, wherein the amount of magnesium is from about 15 to about 1000 parts by weight.

7. The composition of claim 5, wherein the amount of magnesium is from about 100 to about 500 parts by weight.

8. The composition of claim 5, wherein the amount of taurine is from about 50 to about 1500 parts by weight.

9. The composition of claim 5, wherein the amount of taurine is from about 200 to about 600 parts by weight.

10. The composition of claim 5, wherein the amount of carnitine is from about 1 to about 400 parts by weight.

11. The composition of claim 5, wherein the amount of carnitine is from about 100 to about 300 parts by weight.

12. The composition of claim 1, wherein the composition is administered in a pill form.

13. The composition of claim 1, wherein the composition is administered as a liquid.

14. The composition of claim 14, wherein the liquid is an effervescent liquid.

15. An oral dosage treatment composition for ameliorating undesirable side effects due to alcohol consumption comprising:
an effervescent liquid containing a mixture of a magnesium salt, complex, or chelate in an amount of about 15 to about 1000 parts by weight, taurine in an amount of about 50 to about 1500 parts by weight, and carnitine in an amount of about 1 to about 400 parts by weight.

16. A method of ameliorating side effects associated with consumption of alcohol comprising the step of:
administering an oral dosage treatment composition containing a mixture of therapeutically effective amounts of a calcium antagonist, an osmo-regulator, and a fatty acid binder.

17. The method of claim 16, wherein the calcium antagonist is a magnesium salt complex, or chelate.

18. The method of claim 16, wherein the osmo-regulator is taurine.

19. The method of claim 16, wherein the fatty acid binder is carnitine.

20. The method of claim 16, wherein the calcium antagonist is magnesium, the osmo-regulator is taurine, and the fatty acid binder is carnitine.

21. The method of claim 20, wherein the amount of magnesium is from about 15 parts by weight to about 1000 parts by weight.

22. The method of claim 20, wherein the amount of magnesium is from about 100 parts by weight to about 500 parts by weight.

23. The method of claim 20, wherein the amount of taurine is from about 50 parts by weight to about 1500 parts by weight.

24. The method of 20, wherein the amount of taurine is from about 200 parts by weight to about 600 parts by weight.

25. The method of claim 20, wherein the amount of carnitine is from about 1 part by weight to 400 parts by weight.

26. The method of claim 20, wherein the amount of carnitine is 100 parts by weight to 300 parts by weight.

27. The method of claim 16, wherein the composition is administered in a pill form.

28. The method of claim 16, wherein the composition is administered as a liquid.

29. The method of claim 29, wherein the liquid is an effervescent liquid.

30. A method of ameliorating side effects associated with consumption of alcohol comprising the step of:
administering an oral dosage treatment composition containing a mixture of magnesium in an amount of about 15 to about 1000 parts by weight, taurine in an amount of about 50 to about 1500 parts by weight, and carnitine in an amount of about 1 to about 400 parts by weight.
